(19) United States
(12) Patent Application Publication
(10) Pub. No.: US 2017/0246029 A1
(43) Pub. Date: Aug. 31, 2017

(54) PAD, METHOD AND SYSTEM FOR PROVIDING THERMOTHERAPY AT INTRAVASCULAR CATHETER ADMINISTRATION SITE

(71) Applicant: MEDIVANCE INCORPORATED, Louisville, CO (US)

(72) Inventor: Sean Clark, Littleton, CO (US)

(21) Appl. No.: 15/512,025

(22) PCT Filed: Aug. 17, 2015
(86) PCT No.: PCT/US2015/045548

§ 371 Date: Mar. 16, 2017

Related U.S. Application Data
(60) Provisional application No. 62/060,136, filed on Oct. 6, 2014.

(51) Int. Cl. A61F 7/00; A61M 5/14

(52) U.S. Cl. CPC A61F 7/0085; A61M 5/14; A61F 2007/0086; A61F 2007/0056; A61F 2007/0096; A61F 2007/0093

(57) ABSTRACT

An intravascular catheter site pad is provided for thermal exchange (e.g. cooling) adjacent to a catheter introduction site (e.g. a catheter for the administration of a chemotherapeutic agent). The pad includes an opening defined by an opening edge that may be positioned about the catheter introduction site. The pad may further include a fluid containing layer and an inlet port and an outlet port for circulating fluid (e.g. cooled fluid) through the pad. The pad may be fluidly interconnected to a fluid conditioning unit to circulate fluid through the pad under negative pressure. The pad, system and an associated method may be employed to reduce tissue damage at an IV catheter introduction site.

### CROSS-REFERENCE TO RELATED APPLICATION

This application claims priority from U.S. Provisional Application No.: 62/060,136 filed on Oct. 6, 2014, entitled “PAD, METHOD AND SYSTEM FOR PROVIDING THERMOTHERAPY AT INTRAVASCULAR CATHETER ADMINISTRATION SITE,” the contents of which are incorporated by reference herein as if set forth in full.

### FIELD OF THE INVENTION

The present invention relates to a pad, method and system for providing thermotherapy at an intravascular catheter administration site, wherein the invention is particularly apt for use in conjunction with the intravascular administration of chemotherapeutic agents to reduce undesired tissue trauma.

### BACKGROUND OF THE INVENTION

Undesirable tissue reaction attendant to intravascular (IV) catheter administration of medical liquids may occur when the administered liquid escapes from the patient's vein or IV catheter and passes into subcutaneous or subdermal tissues surrounding the administration site. In particular, undesired tissue reactions are not unusual in relation to the administration of chemotherapeutic agents utilized in the treatment of cancer. Such chemotherapeutic agents may be characterized as irritants and vesicants. Of particular concern are vesicants which may cause serious administration site reactions, sometimes referred to as chemical cellulitis. Such vesicants can cause severe tissue damage, dependent upon the vesicant potential of the chemotherapeutic agent, the amount and concentration of chemotherapeutic agent exposure, and mitigating measures taken once extravasation occurs.

In the latter regard, tissue damage mitigation measures have been proposed which include the application of ice packs to an IV administration site, most typically after administration of a chemotherapeutic agent. Unfortunately, such mitigation measures often yield insufficient benefit in limiting tissue damage.

### SUMMARY OF THE INVENTION

In view of the foregoing, inventive thermotherapy modalities are described herein to reduce tissue damage at intravascular IV catheter administration sites.

In one embodiment, a flexible pad is provided for contact and thermal exchange with a patient adjacent to an IV catheter administration site. The pad may comprise a fluid containing layer for containing a fluid circulatable therethrough (e.g. a cooled liquid such as water), and an inlet port and outlet port, each fluidly interconnected to the fluid containing layer for flowing the circulatable fluid in to and out of the fluid containing layer. The fluid containing layer may be at least partially defined by and between flexible, first and second layers. The pad may further include an opening extending through and defined by an opening edge of the pad, wherein the opening may be configured so that the opening edge is positionable about at least a portion of the IV catheter administration site, and wherein the circulatable fluid is flowable about at least a portion of the opening to provide for thermal exchange with the patient (i.e. thermal exchange between the circulated fluid and the patient). By way of particular example, the circulatable fluid may be cooled to provide for contact cooling of a patient about the IV catheter administration site (e.g. via transdermal thermal exchange) in conjunction with the administration of a medical liquid (e.g. a chemotherapeutic agent) via an IV catheter at the IV catheter administration site.

In some implementations, the pad may further comprise an adhesive surface disposed on a skin contacting side of the fluid containing layer, wherein the pad is directly adherable to the patient by directly contacting the adhesive surface with skin of the patient, and thermal energy is exchangeable between the circulatable fluid and the patient across the adhesive surface. In that regard, the pad may be conformable and adherable to the patient to facilitate thermal exchange. The adhesive surface may be provided to extend at least partially about, and in some applications substantially entirely about, the opening of the pad. As may be appreciated, the adhesive surface may provide for intimate skin contact, thereby enhancing thermal exchange between the circulatable fluid and the patient (e.g. by reducing “tenting” of the pad over skin portions). Further, the adhesive surface, together with the interconnected first and second layers, may present a physical barrier to contain any medical liquid that may escape during an IV catheter administration procedure. In that regard, such containment may reduce the area of undesired contact between an administered liquid (e.g. a chemotherapeutic agent) and patient tissue.

In some embodiments, the adhesive surface may be defined by a flexible thermally conductive hydrogel layer. For example, the hydrogel layer may comprise a matrix of a polymer material and water.

In some implementations, the opening may be configured so that the opening edge is positionable substantially entirely about an IV catheter administration site. For example, the pad may include a slit that extends through the pad from an outer peripheral edge of the pad to adjoin the opening, wherein a cross-dimension of the opening is greater than a cross-dimension of the slit, and wherein the pad may be positioned with the IV catheter introduction site exposed through the opening. This approach facilitates pad positioning (e.g. prior to or after transcutaneous positioning of an IV catheter at the IV catheter administration site) and repositioning of the pad (e.g. after transcutaneous positioning of the IV catheter).

In other implementations, the opening may be configured as a recess along an outer edge of the pad. In further implementations, the opening may be configured as a hole through the pad, wherein the opening edge extends 360° to define the hole.

In some arrangements, the opening may be of an elongate configuration having a maximum length dimension that is greater than a maximum width dimension. In turn, the pad may positionable so that a center axis of the opening along the length dimension is substantially aligned with a vein of the patient. Correspondingly, in use of the pad, an IV catheter may be introduced in to the vein of the patient, in substantially aligned relation to the center axis of the opening and the vein.

Further, the opening may be located closer to one outer edge portion of the pad than other outer edge portions of the pad. In one example, the pad may be of a rectangular configuration, wherein the opening is positioned closer to a first side of the pad than the other three sides of the pad. Further, the opening may be centered on a center axis of the pad that extends parallel to and between a second side and a third side of the pad that each adjoin the first side.

Optionally, the fluid containing layer of the pad embodiment may comprise a plurality of channels for directing the flow of the circulatable fluid between the inlet port and the outlet port. In turn, at least a portion of at least one of the plurality of channels may extend about at least a portion of the opening. In one approach, the inlet port may be disposed to flow the circulatable fluid into a first end of each of the plurality of channels, and the outlet port may be disposed to flow the circulatable fluid out of a second end of each of the plurality of channels. In some implementations, the inlet port and the outlet port may comprise corresponding first ends that interface the fluid containing layer at laterally-offset locations. Further, the inlet port and the outlet port may comprise corresponding second ends that extend laterally outside of the fluid containing layer in aligned, stacked relation to one another and in laterally-offset, parallel relation to the center axis of the opening.

In a system embodiment, a fluid circulation unit may be fluidly interconnected via fluid circulation lines to a contact pad having one or more of the above-referenced features, wherein the fluid circulation unit is operable to circulate fluid through the fluid circulation lines and the pad for sustained contact thermal exchange with a patient (e.g. at an IV catheter administration site). In that regard, the fluid circulating unit may comprise a fluid reservoir for containing a circulatable fluid (e.g. a liquid such as water) and a fluid circulation pump, wherein upon operation of the fluid circulation pump fluid is drawn through the pad from the fluid reservoir (e.g. at a negative pressure) then pumped by the circulation pump back in to the fluid reservoir. The fluid circulation unit may also include a heat exchanger interconnected to the fluid reservoir for use in controlling a temperature of the circulated fluid. In particular, the heat exchanger may be provided to cool the fluid in the fluid reservoir. The cooled fluid may be circulated through the pad to provide sustained contact cooling of a patient at an IV catheter administration site.

The fluid circulation unit may further include a controller for controlling operation of the heat exchanger and fluid circulation pump. In that regard, the controller may be provided so as to provide for temperature control of the circulated fluid in a predetermined manner. For example, the controller may provide control signals to control the operation of the heat exchanger so as to cool the circulated fluid and maintain the circulated fluid at a predetermined temperature, e.g. a temperature that may be selectively established by a user. In that regard, the system embodiment may further include at least one fluid temperature sensor for sensing a temperature of the circulated fluid (e.g. a temperature of the fluid within the fluid reservoir) and for providing a fluid temperature signal indicative thereof. In turn, the controller may be provided to utilize the fluid temperature signal in providing control signals to the heat exchanger.

Further, in some implementations, the controller may further provide control signals to control the operation of the heat exchanger so as to cool the circulated fluid and thereby cool and/or otherwise maintain (i.e. via the contact pad) the skin region of a patient adjacent to an IV catheter administration site at a temperature within a predetermined temperature range (e.g. during the administration of a medical liquid such as a chemotherapeutic agent). In that regard, the system embodiment may further include a patient temperature sensor for sensing a temperature of the skin region adjacent to the IV catheter administration site (e.g. adjacent to or under the contact pad) and for providing a patient temperature signal indicative thereof. In turn, the controller may be provided to utilize the patient temperature signal in providing control signals to the heat exchanger.

In addition to the foregoing, a method embodiment is provided for contact thermal exchange between a pad and a patient adjacent to an IV catheter administration site. The embodiment includes the step of positioning a pad (e.g. a pad having one or more of the features described above) in contact with a patient, wherein an opening of the pad that is defined by an opening edge of the pad is positioned so that the opening edge extends about at least a portion of an IV catheter administration site. The method may further include the steps of locating transcutaneously an IV catheter in to the patient's vascular system at the IV catheter administration site, administering a medical liquid (e.g. a chemotherapeutic agent) through the IV catheter after the locating step, and circulating a fluid (e.g. a liquid such as water) through a fluid containing layer of the pad during at least a portion of the administering step. In conjunction with the circulating step, the circulated fluid may flow about at least a portion of the opening for transdermal thermal exchange with the patient. In that regard, the method may further provide for cooling the fluid circulated through the fluid containing layer to provide contact cooling of the patient adjacent to the IV catheter administration site during the circulating step, thereby reducing potential tissue damage attendant to administration of a chemotherapeutic agent.

In some arrangements, the method embodiment may include the additional steps of sensing a temperature of a skin region adjacent to the IV catheter administration site, and providing a patient temperature signal indicate thereof. In turn, the patient temperature signal may be utilized in the controlling step.

Optionally, the circulating step may be initiated prior to the administering step, e.g. so as to cool a tissue region adjacent to the IV catheter introduction site (e.g. cooling to a predetermined temperature as sensed by the patient temperature sensor). Further, the circulating step may be continued during a portion of, or during the entirety of, the administering step, wherein the tissue region may be maintained (e.g. as sensed by the patient temperature sensor) at a temperature within a predetermined temperature range.

In some implementations, the positioning step may be completed so that the pad is disposed in fixed relation to the IV catheter administration site. In one approach, the positioning step may entail adhering an adhesive surface of the pad to skin of the patient to yield fixed positioning. In that regard, the adhesive surface may be provided so that thermal energy is exchangeable between the circulated fluid and the patient across the adhesive surface. Further, the adhesive surface may be provided to have a peel value of between about 10 to 200 gm/inch, and preferably between about 20 to 80 gm/inch, thereby facilitating fixed positioning, repositioning and removal of the pad.

In certain embodiments, the adhesive surface may be defined by a thermally conductive hydrogel layer that extends across at least a portion of a skin contacting side of the fluid containing layer of the pad. Preferably, the thermally conductive hydrogel layer may extend across at least a majority, or even the entirety, of the skin contacting side of the fluid containing layer.

In some implementations, the adhering step may include the sub-steps of first adhering a first portion of the adhesive surface that extends at least partially about the opening adjacent to the IV catheter administration site, and second adhering a second portion of the adhesive surface. Further, the method may include the step of removing at least a first portion of a removable liner from at least the first portion of the adhesive surface prior to the first adhering step. By way of example, the first portion of the removable liner may be peeled away from the first portion of the adhesive surface, thereby allowing the first portion of the adhesive surface to be adhered to a patient in a desired location (e.g. adjacent to one side of an IV catheter administration site). Then, a second portion of the removable liner may be pulled away from a second portion of the adhesive surface, thereby allowing the second portion of the adhesive surface to be adhered to the patient (e.g. adjacent to another side of an IV catheter administration site).

In some embodiments the opening of the pad may be of an elongate configuration with a maximum length dimension greater than a maximum width dimension, wherein the positioning step may include locating the pad so that a center axis of the opening extending along the length dimension is substantially aligned with a vein of the patient. In some embodiments, the locating step may comprise introducing the intravascular catheter into the vein of the patient in aligned relation to the center axis of the opening and the vein of the patient.

In contemplated embodiments, the method may further include the step of controlling the temperature of the circulated fluid in a predetermined manner during at least a portion of the circulating step. In one approach, the controlling step may include controlling the operation of a heat exchanger to provide for selective cooling and optional heating of the circulated fluid. In some arrangements, such selective cooling may be implemented so as to cool the circulated fluid to a predetermined temperature and/or to otherwise maintain the temperature of the circulated fluid within a predetermined range.

As may be appreciated, features of the pad, system and method embodiments described herein may be used in combination. Numerous additional features and advantages of the present invention will become apparent to those skilled in the art upon consideration of the embodiment descriptions provided hereinbelow.

### BRIEF DESCRIPTION OF THE DRAWINGS

FIG. 1 is a perspective view of one embodiment of a pad for contact and thermal exchange with a patient adjacent to an intravascular IV catheter administration site. FIG. 2 illustrates multiple layers comprising the pad embodiment of FIG. 1.

FIG. 3 illustrates the pad embodiment of FIG. 1 positioned on an arm of a patient adjacent to an IV catheter administration site.

FIG. 4 illustrates a system embodiment in which the pad embodiment of FIG. 1 is fluidly interconnected to a fluid circulation unit.

FIG. 5 illustrates a method embodiment for use of a pad to for contact and thermal exchange with a patient adjacent to an IV catheter administration site during administration of a medical liquid (e.g. a chemotherapeutic agent).

### DETAILED DESCRIPTION

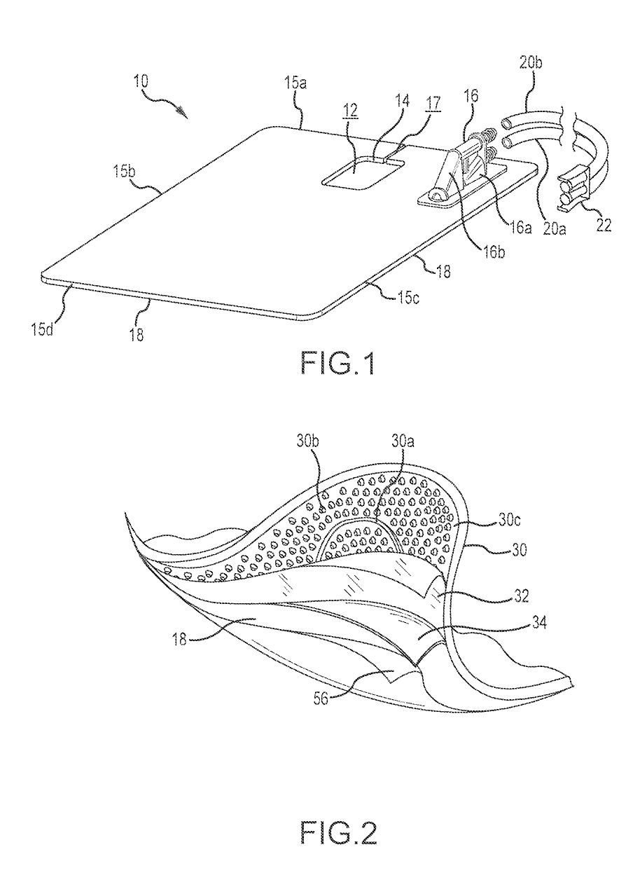

One embodiment of a pad 10 for contact and thermal exchange with a patient adjacent to an intravascular (IV) catheter administration site is illustrated in FIGS. 1 and 2. The IV site pad 10 may include an opening 12 extending through the IV site pad 10, wherein the opening 12 is defined by an opening edge 14 of the IV site pad 10. The opening 12 may be provided so that the opening edge 14 may be positioned about at least a portion of an IV catheter introduction site. Optionally, a slit 17 through the IV site pad 10 may be provided and may extend from opening 12 to side 15a of the IV site pad 10. The slit 17 facilitates positioning of the IV site pad 10 relative to an IV catheter administration site either prior to or after transcutaneous positioning of an IV catheter at the IV catheter administration site.

Additionally, the IV site pad 10 may comprise an adhesive surface 18 on a skin-contacting side thereof for adhering the IV site pad 10 in fixed relation to an IV catheter administration site. The adhesive surface may extend across at least a portion, and in some embodiments across a majority or an entirety, of the skin-contacting side of the IV site pad 10.

The IV site pad 10 may further include an inlet port and an outlet port for circulating fluid (e.g. a liquid such as water) in to and out of a fluid containing layer of the IV site pad 10. In the embodiment of FIG. 1, the inlet port and outlet port are defined by a dual port manifold 16. The dual port manifold 16 defines an inlet port 16a and an outlet port 16b having corresponding first ends that interface with the fluid containing layer of the IV site pad 10 at laterally-offset locations. The inlet port 16a and outlet port 16b further include corresponding second ends that extend laterally outside of the fluid containing layer in aligned, stacked relation to one another. As illustrated, the second ends of inlet port 16a and outlet port 16b may be configured (i.e. with a plurality of annular barbs) for fixed interconnection with fluid circulation lines 20a and 20b, respectively. In one approach, the fluid circulation lines 20a and 20b may be defined by lengths of flexible tubing. The fluid circulation lines 20a, 20b may be provided with a connector 22 for use in selective interconnection to and disconnection from a fluid circulation unit, wherein fluid may be circulated through the IV site pad 10, as will be further described hereinbelow.

As illustrated in FIG. 2, the IV site pad 10 may include a flexible first layer 30 and flexible second layer 32 that are peripherally interconnected to define the fluid containing layer of IV site pad 10 therebetween. Further, IV site pad 10 may comprise a flexible third layer 34 interconnected to the second layer 32 and defining the adhesive layer 18. A removable fourth layer 36 may also be provided to cover the adhesive layer 18 prior to use.

The first layer 30 may comprise one or a plurality of fluid channels. In that regard, the first layer 30 may include one or a plurality of rib members 30a that are interconnected to the second layer 32. The fluid channels may extend between adjacent rib members 30a and/or between sealed edges of the IV site pad 10 and/or between rib members 30a and sealed edges of the IV site pad 10. The rib members 30a may be configured to direct the flow of fluid between the inlet port 16a and outlet port 16b.

At least a portion of one or more of the fluid channels may extend about at least a portion of the opening 12. Further, the fluid channels may be configured to provide for fluid flow across the lateral extent of the IV site pad 10. In some embodiments, the inlet port 16a and fluid channels may be spaced to define a staging region within the fluid containing layer that is adjacent to and fluidly interconnected to a first end of each of a plurality of channels. Further, the outlet port 16b and fluid channels may be spaced to define another staging region within the fluid containing layer that is adjacent to and fluidly interconnected to a second end of each of a plurality of channels.

As further illustrated in FIG. 2, the first layer 30 may also comprise a plurality of offset projections 30b, or inverted dimples. The projections 30b may be provided to supportably engage the second layer 32, thereby maintaining and defining tortuous fluid flow passageways through the fluid channels of the fluid containing layer. In that regard, and as will be further described, in system embodiments fluid may be circulated through the fluid containing layer of the IV site pad 10 at negative pressure, wherein the projections 30b keep the second layer 32 from collapsing across the first layer 30, thereby maintaining fluid flow.

In one embodiment, the first layer 30 may be defined by a closed foam material (e.g. a polymer foam material) that is heat pressed to form the rib members 30a and inverted dimples 30b that project away from a base portion 30c of the first layer 30. The second layer 32 may comprise a heat activatable film (e.g. a polymer material) that may be sealably bonded about its periphery to the periphery of the first layer 30. Further, the heat lamination process may bond the second layer 32 to interfacing surfaces of the rib members 30a, and optionally to interfacing surfaces of the projections 30b.

In some embodiments, the third layer 34 may comprise a thermally-conductive hydrogel layer that may be applied to the second layer 32 by adhesion. The hydrogel layer may comprise a matrix of water and a polymer material.

In some embodiments, the removable fourth layer 36 (e.g. a release liner) may be provided to peel away from adhesive surface 18. In that regard, successive portions of the fourth layer 36 may be pulled away from adhesive 18 to allow for successive adhesive positioning of different portions of adhesive surface 18 at an IV catheter administration site.

In one implementation, the dual port manifold 16 that defines the inlet port 16a and the outlet port 16b may be heat bonded to the first layer 30 over corresponding inlet and outlet holes that are cut through the first layer 30 prior to interconnection of the first layer 30 to the second layer 32. As may be appreciated, such inlet and outlet holes allow for the circulation of fluid in to and out of the fluid containing layer of the IV site pad 10.

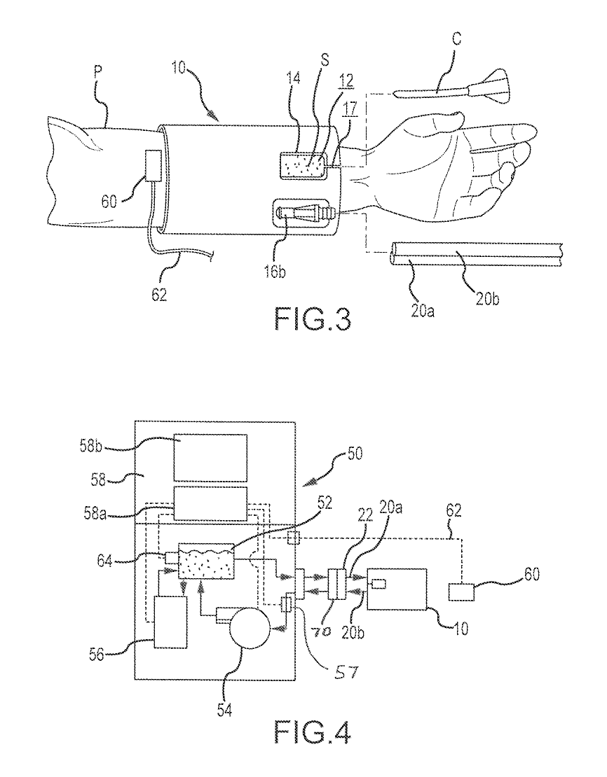

FIG. 3 illustrates the IV site pad 10 positioned on the arm of a patient P. As may be appreciated, the IV site pad 10 may be utilized at other body locations as well, including for example the leg, shoulder or hip of a patient. In that regard, the illustrated configuration of IV site pad 10 facilitates such alternative site applications.

As shown in FIG. 3, the IV site pad 10 may be positioned so that the opening edge 14 extends substantially entirely about an IV catheter administration site (S). Optionally, in some implementations, the IV catheter introduction site S may be identified, or indicated, by medical personnel prior to the transcutaneous introduction of an IV catheter C at the IV catheter introduction site S, and optionally, prior to or after positioning the IV pad site 10 on the patient P. For example, a disinfectant or other material that is visually discernible may be applied to the IV catheter administration site S.

To position IV site pad 10 on the patient P, the fourth layer 36 of the IV site pad 10 may be removed so as to successively expose adjacent portions of the adhesive surface 18. In turn, the IV site pad 10 may be positioned so as to progressively adhere the adhesive surface 18 across a skin region of the patient P with opening 12 located to provide access to the IV catheter administration site.

As may be appreciated, slit 17 particularly facilitates positioning of the IV site pad 10 after an IV catheter at the IV catheter introduction site S has been transcutaneously introduced at the IV catheter C administration site S. In one approach, a first portion of the removable layer 36 on a first side of the slit 17 (e.g. on a side nearest side 15b of IV site pad 10) may be pulled away to expose a corresponding first portion of adhesive surface 18, wherein such first portion of adhesive surface 18 may be readily adhered to a first skin region adjacent to IV catheter administration site S (e.g. by progressively laying down the first portion). Thereafter, a second portion of the removable layer 36 on a second side of the slit 17 (e.g. on a side nearest side 15c of IV site pad 10) may be pulled away to expose a corresponding second portion of adhesive surface 18, wherein the second portion of adhesive surface 18 may be readily adhered to a second skin region adjacent to IV catheter administration site S (e.g. by progressively laying down the second portion).

The third layer 34 of the IV site pad 10 may be provided so that adhesive surface 18 has a peel value of between about 10 to 200 gm/inch, and preferably between about 20 to 80 gm/inch, thereby facilitating fixed positioning, repositioning and removal of the IV site pad 10 adjacent to the IV catheter administration site S.

In the embodiment shown in FIGS. 1 and 3, the opening 12 of IV pad site 10 may be configured so that the opening edge 14 is positionable substantially entirely about an IV catheter administration site S. For such purposes, apart from slit 17, the opening edge 14 may be continuous about the opening 12.

The opening 12 may be located off-center, closer to a given peripheral edge portion than other edge portions of IV site pad 10. For example, in the illustrated embodiment, IV site pad 10 is of a rectangular configuration and opening 12 is located closer to side 15a of the IV site pad than the other three sides 15b, 15c and 15d of the IV site pad 10. Further, opening 12 may be positioned mid-way between side edge 15b and side edge 15c. For example, as shown in FIG. 1, opening 12 and slit 17 may be centered on a center axis of IV site pad 10.

As further shown in FIGS. 1 and 3, the opening 12 may be of an elongate configuration (e.g. rectangular) with a maximum length dimension that is greater than a maximum width dimension. Further, the maximum width dimension of opening 12 may be greater than, e.g., at least 2 times greater than, a maximum width dimension of slit 17.

Pad 12 may be positionable so that a center axis of opening 12 along the length dimension is substantially aligned with a vein of a patient. Additionally, slit 17 may be substantially aligned with such center axis. Correspondingly, an intravascular catheter may be introduced into the vein of a patient, in substantially aligned relation to the center axis of the opening and the vein. Further, shown in FIGS. 1 and 3, the dual port manifold 16 may be provided so that the second ends of the inlet port 16a and outlet port 16b extend in laterally-offset, parallel relation to the center axis of the opening 12. Additionally, the dual port manifold 16 may be located so that the second ends of inlet port 16a and outlet port 16b may extend towards side 15a, wherein circulation lines 20a, 20b may be interconnected to inlet port 16a and outlet port 16b to conveniently extend away from the IV site pad 10 in a direction that avoids interference with the IV catheter administration site S.

Reference is now made to FIG. 4 which schematically illustrates a system embodiment in which IV site pad 10 may be fluidly interconnected to fluid circulation lines 22a, 22b which may be selectively, fluidly interconnected to a fluid circulation unit 50. The fluid circulation unit 50 may include a fluid reservoir 52 that contains a fluid (e.g. water) and that is fluidly interconnectable to fluid circulation line 22a. The fluid circulation unit 50 may also include a fluid circulation pump 54 that is fluidly interconnectable to fluid circulation line 22b. For purposes of fluidly interconnecting fluid circulation lines 20a, 20b with fluid circulation unit 50, the connecter 22 may be configured for selective connection to and disconnection from a compatible connector 70 provided on a reusable hose assembly that is interconnectable to and disconnectable from fluid circulation unit 50. In that regard, connectors may be employed as taught in U.S. Pat. No. 6,802,855, hereby incorporated by reference in its entirety.

Upon operation of the fluid circulation pump 54, fluid is drawn through IV site pad 10 from fluid reservoir 52 (e.g. at a negative pressure) and pumped by circulation pump 54 back into fluid reservoir 52. As shown in FIG. 4, fluid circulation unit 50 may also include a heat exchanger 56 fluidly interconnected to the fluid reservoir 52 for use in controlling a temperature of the circulated fluid. In particular, heat exchanger 56 may be provided to cool the fluid in fluid reservoir 52. In turn, the cooled fluid may be circulated through the IV site pad 10 to provide contact cooling of a patient at an IV catheter administration site (e.g. during administration of a chemotherapeutic agent). Optionally, heat exchanger 56 may be further provided to warm, or rewarm, the circulated fluid.

As shown in FIG. 4, the fluid circulation unit 50 may further include a controller 58 for controlling operation of the heat exchanger 56 and fluid circulation pump 54. The controller 58 may be computer-based (e.g., a microprocessor) and may include a programmable control module 58a and a user interface 58b for receiving user control input and for providing corresponding signals to the programmable control module 58b.

As shown in FIG. 4, fluid circulation unit 50 may also include a fluid temperature sensor 64 for sensing a temperature of the circulated fluid in fluid reservoir 52 and for providing a fluid temperature signal indicative of the sensed temperature to controller 58a. The controller 58a may utilize the fluid temperature signal in providing control signals to heat exchanger 56, wherein the control signals may control heat exchanger 56 to provide a predetermined magnitude of fluid cooling, and optionally a fluid warming. In one approach, the controller 58a may utilize the fluid temperature signal to provide control signals to heat exchanger 56 to cool the circulated fluid to a predetermined temperature and/or to otherwise maintain the circulated temperature within a predetermined temperature range.

Further, control signals may be provided by controller 58a to fluid circulation pump 54 (e.g. control signals to control a speed or fluid pumping rate of fluid circulation pump 54). In that regard, fluid circulation unit 50 may further include a pressure sensor 57 for sensing a fluid stream pressure upstream of the fluid circulation pump 54 and providing a fluid pressure signal to controller 58a that is indicative of the sensed fluid stream pressure. In turn, controller 58a may utilize the sensed fluid stream pressure signal in providing control signals to the circulation pump 54 (e.g. to control the speed or fluid pumping rate so as to maintain a desired negative pressure within IV site pad 10).

As shown in FIGS. 3 and 4, the system embodiment may further include a patient temperature sensor 60 for sensing the temperature of a skin region adjacent to an IV catheter administration site S and for providing a patient temperature signal indicative thereof via signal line 62. For example, patient temperature sensor 60 may comprise a thermostat that may be fixedly/removably positioned on the skin (e.g. via tape or adhesive backing) in a location that overlies a tissue region that is downstream of the IV catheter.

The controller 58a may be provided to utilize the patient temperature signal in providing control signals to the heat exchanger 56. In one approach, the controller 58a may utilize the patient temperature signal to provide control signals so as to control the cooling of the circulated fluid and thereby cool and/or otherwise maintain the skin region adjacent to an IV catheter administration site at a temperature within a predetermined range. For example, in some embodiments, the heat exchanger 56 may be controlled to initially cool the skin region to a predetermined temperature (e.g. as determined by the controller 58a using the patient temperature signal) prior to the administration of a medical liquid (e.g. a chemotherapeutic agent) via an IV catheter C at the IV catheter administration site S. Further, the control signals may be provided to control the heat exchanger 56 so as to maintain the temperature of the skin region (e.g. as determined by the controller 58a using the patient temperature signal) within a predetermined temperature range during the medical liquid administration procedure. As may be appreciated, the degree of skin cooling may be established so as to effect a degree of cooling by IV site pad 10 to reduce undesired tissue damage at the IV catheter administration site S during the administration of the medical liquid.

It is believed that contact cooling by IV site pad 10 may cause tissue contraction which may reduce undesired tissue penetration of a medical liquid. Additionally, or alternatively, it is believed that contact cooling by IV site pad 10 may effectively suspend undesired operative effects of a medical liquid, e.g. the cooling may suspend the heating effects of chemotherapeutic agents.

With further reference to FIG. 4, the programmable control module 58a may be provided to store control data (e.g., via a computer readable medium) and generate control signals in corresponding relation to a plurality of different temperature control phases. In that regard, the programmable control module 58a may comprise control logic for utilizing the control data to provide control signals to the heat exchanger 56 and/or the fluid pump 54, wherein the temperature of the circulated fluid may be controlled in a predetermined manner for each of the plurality of different temperature control phases. Additionally or alternatively, the programmable control module 58a may be provided to facilitate the establishment of one or more programmed protocols that each comprise control data for use in the control of each of the plurality of temperature control phases. By way of example, a given protocol may comprise control data that includes target temperature data for each of a plurality of treatment phases. For example, the target temperature data may comprise target skin temperatures for a patient skin region adjacent to an IV catheter administration site S. Further, for one or more of the phases, the protocol may comprise control data comprising a set duration for thermal treatment. As may be appreciated, the user interface 58b may be adapted for use in receiving user input to establish the control data corresponding with each of the plurality of different temperature control phases on a protocol-specific basis.

For each given protocol the programmable control module 58a may provide control signals to at least the heat exchanger 56, and optionally to fluid pump 54, on a phase-specific basis. In turn, heat exchanger 56 may be provided to responsively change the temperature of the circulated fluid to affect a desired thermal exchange with a patient (i.e. adjacent to IV introduction site S), e.g., to cool, maintain the temperature of, or warm tissue via IV site pad 10.

Optionally, the user interface 58b may be provided to include a graphic display to visually present a plot of a target skin temperature that is based on the stored control data for a plurality of different temperature control phases. Further, the graphic display may be operable to display a plot of a sensed patient skin temperature (e.g., as sensed by patient temperature sensor 60) in corresponding time relation to the plot of the target skin temperatures. Further, the graphic display may be operable to display a plot of a sensed temperature of the circulated fluid (e.g. as sensed by fluid temperature sensor 64) in corresponding time relation to the plot of the target temperature adjustment rate.

In one example, the fluid circulation unit 50 may utilize the Arctic Sun 5000 Temperature Management System product of Medivance, Inc., located in Louisville, Colo., USA.

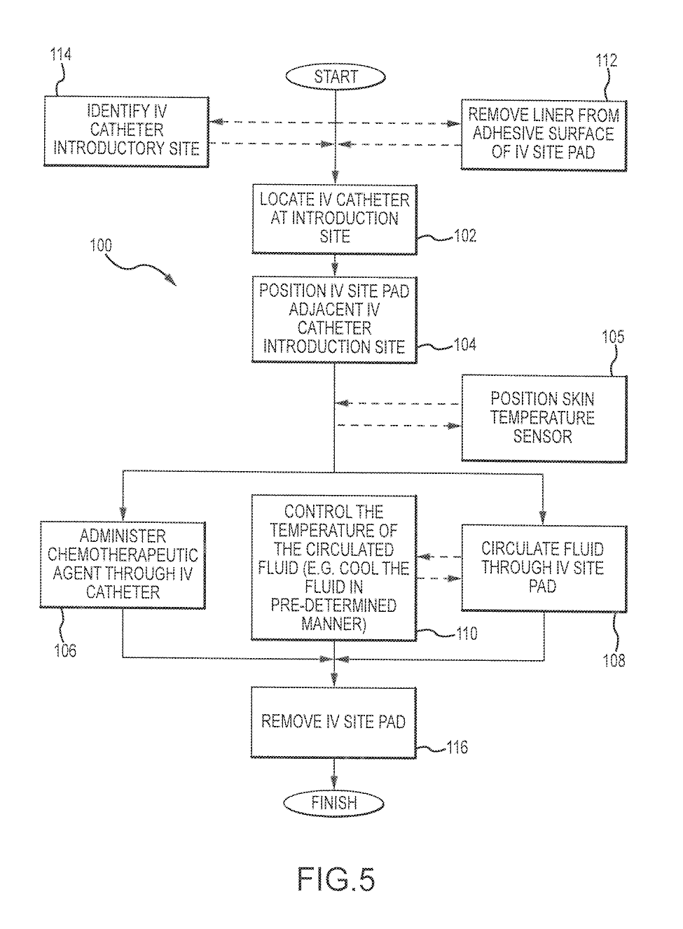

FIG. 5 illustrates steps of a method embodiment 100 for contact thermal exchange between a pad and a patient at an IV catheter administration site (e.g. adjacent thereto). The illustrated embodiment includes the step of locating transcutaneously an IV catheter into the patient's vascular system (e.g. a vein of the patient) at the IV catheter administration site (step 102). Further, the embodiment includes the step of positioning an IV site pad adjacent to the IV catheter administration site (step 102). In particular, the positioning step may include positioning of the IV site pad 10 described above, wherein opening 112 is positioned so that the opening edge 114 extends about at least a portion of an IV catheter administration site S as described above. Steps 102 and 104 may be completed in either order, i.e. step 102 then step 104, or step 104 then step 102.

Further, the method embodiment may include the steps of administering a medical liquid (e.g. a chemotherapeutic agent) through the IV catheter (step 106), and circulating a fluid through a fluid containing layer of the IV site pad 10 during at least a portion of the administering step (step 108). By way of example, the circulating step may be completed utilizing the fluid circulation unit 50 described above. The circulating step 108 may be completed so that the circulated fluid flows about at least a portion of opening 12 of IV site pad 10 for transdermal thermal exchange with the patient.

The method embodiment 100 may further include the step of controlling the temperature of the circulated fluid (step 110). In particular, the method may provide for cooling the circulated fluid to provide for contact cooling of the patient adjacent to the catheter introduction site during the circulating step 108. For temperature control purposes, a skin temperature sensor (e.g. temperature sensor 60) may be positioned adjacent to the IV catheter administration site (step 105) and may provide a patient temperature signal for use in controlling the temperature of the circulated fluid, as described above.

Optionally, the positioning step 102 may include adhering an adhesive surface of the IV site pad to the skin of the patient, wherein thermal energy is exchanged between the circulated fluid and the patient across the adhesive surface. In that regard, the method may further include the step of removing a liner from adhesive surface 18 of IV site pad 20 (step 112), prior to the IV site pad positioning step 102. The method embodiment may optionally also include the step of identifying the IV catheter introductory site (step 114), prior to the IV site pad positioning step 102. Additional method steps may be provided in corresponding relation to the IV site pad 10, fluid circulation unit 50 and system embodiments described hereinabove.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain known modes of practicing the invention and to enable others skilled in the art to utilize the invention in such or other embodiments and with various modifications required by the particular application(s) or use(s) of the present invention. It is intended that the appended claims be construed to include alternative embodiments to the extent permitted by the prior art.

What is claimed is:

1. A pad for contact and thermal exchange with a patient, comprising:
a fluid containing layer for containing a fluid circulatable therethrough;
an inlet port and an outlet port, each fluidly interconnected to said fluid containing layer for flowing said circulatable fluid in to and out of said fluid containing layer; and
an opening extending through and defined by an opening edge of said pad, wherein said opening is configured so that said opening edge is positionable about at least a portion of an intravascular catheter administration site, wherein said circulatable fluid is flowable about at least a portion of said opening for thermal exchange with the patient.

2. The pad of claim 1, further comprising:
an adhesive surface disposed on a skin contacting side of the fluid containing layer, wherein the pad is directly adherable to the patient by directly contacting the adhesive surface with skin of the patient and thermal energy is exchangeable between the circulatable fluid and the patient across the adhesive surface.

3. The pad of claim 2, wherein the adhesive surface extends at least partially about the opening.

4. The pad of claim 2, wherein the adhesive surface is defined by a thermally-conductive hydrogel.

5. The pad of claim 1, wherein the opening is configured so that said opening edge is positionable substantially entirely about said intravascular catheter introduction site.

6. The pad of claim 5, further comprising:
a slit extending through the pad from an outer peripheral edge of the pad to said opening.

7. The pad of claim 1, wherein the opening is of an elongate configuration having a maximum length dimension that is greater than a maximum width dimension, and wherein said pad is positionable so that a center axis of the opening along said length dimension is substantially aligned with a vein of the patient.

8. The pad of claim 1, said fluid containing layer comprising:
a plurality of channels for directing the flow of said circulatable fluid between the inlet port and the outlet port, wherein at least a portion of at least one of the plurality of channels extends about at least a portion of the opening.

9. The pad of claim 8, wherein the inlet port is disposed for flow of the circulatable fluid in to a first end of each of the plurality of channels, and wherein the outlet port is disposed for flow of the circulatable fluid out of a second end of each of the plurality of channels.

10. The pad of claim 9, wherein the first port and the second port comprise corresponding first ends that interface the fluid containing layer at laterally-offset locations, and wherein the first port and the second port comprise corresponding second ends that extend laterally outside the fluid containing layer in aligned, stacked relation.

11. A method for contact thermal exchange between a pad and the patient, comprising:
locating transcutaneously an intravascular catheter in to a patient's vascular system at an intravascular catheter administration site;
positioning a pad in contact with a patient, wherein an opening of the pad that is defined by an opening edge of the pad is positioned so that the opening edge extends about at least a portion of the intravascular catheter administration site;
administering a medical liquid through an intravascular catheter at the intravascular catheter administration site after said locating step and positioning step; and
circulating a fluid through a fluid containing layer of the pad during at least a portion of the administering step, wherein the fluid flows about at least a portion of the opening for transdermal thermal exchange with the patient.

12. The method of claim 11, further comprising:
cooling the circulated fluid to provide contact cooling of the patient adjacent to the catheter administration site during said circulating step.

13. The method of claim 11, wherein in said positioning step said pad is disposed in fixed relation to the patient.

14. The method of claim 13, wherein said positioning step comprises:
adhering an adhesive surface of the pad to skin of the patient, wherein thermal energy is exchangeable between the circulated fluid and the patient across the adhesive surface.

15. The method of claim 14, wherein the adhesive surface is defined by a thermally-conductive hydrogel layer that extends across at least a majority of a skin contacting side of the fluid containing layer of the pad.

16. The method of claim 14, wherein the adhering step comprises:
first adhering a first portion of the adhesive surface that extends at least partially about the opening adjacent to the intravascular catheter administration site; and
second adhering a second portion of the adhesive surface.

17. The method of claim 16, further comprising:
removing at least a first portion of a removable liner from at least the first portion of the adhesive surface prior to said first adhering step.

18. The method of claim 11, wherein the opening of the pad is of an elongate configuration having a maximum length dimension that is greater than a maximum width dimension, and wherein said positioning step comprises:
positioning the pad so that a center axis of the opening along the length dimension is substantially aligned with a vein of the patient.

19. The method of claim 18, wherein the locating step comprises:
introducing the intravascular catheter in to the vein of the patient in aligned relation to the center axis of the opening and the patient vein.

20. The method of claim 11, further comprising:
controlling a temperature of the circulated fluid in a predetermined manner during at least a portion of the circulating step.

21. The method of claim 20, wherein said circulating step comprises:
operating a fluid pump to circulate the fluid from a fluid reservoir through the fluid containing layer and back into the fluid reservoir, wherein a heat exchanger is disposed in fluid communication with fluid reservoir.

22. The method of claim 21, further comprising:
positioning a patient temperature sensor on the patient to sense a temperature of a skin region adjacent to the intravascular catheter administration site, wherein the patient temperature sensor provides a patient temperature signal indicative of the sensed temperature.

23. The method of claim 22, wherein the controlling step comprises:
utilizing the patient temperature signal at a controller to provide control signals to control the heat exchanger.

24. The method of claim 23, wherein the controlling step further comprises:
utilizing a fluid temperature signal indicative of a temperature of the circulated fluid to provide said control signals to the heat exchanger.

25. The method of claim 24, wherein said heat exchanger cools said circulated fluid in response to said control signals.

26. The method of claim 25, wherein said controller provides said control signals to the heat exchanger so as to maintain a temperature of said skin region at a temperature within a predetermined temperature range.

27. The method of claim 20, wherein the controlling step comprises:
cooling the circulated fluid during at least a portion of the circulating step, wherein the cooling step is initiated prior to the administering step to cool a skin region at the pad to a predetermined temperature.

28. The method of claim 27, wherein the controlling step is completed to maintain a temperature of the skin region within a predetermined temperature range throughout the administering step.

29. The method of claim 11, wherein the medical liquid is a chemotherapeutic agent.

30. The method of claim 29, wherein the controlling step comprises:
cooling the circulated fluid during at least a portion of the circulating step, wherein the cooling step is initiated prior to the administering step to cool a skin region at the pad to a predetermined temperature, and wherein the controlling step is completed to maintain a temperature of the skin region within a predetermined temperature range throughout the administering step.

31. A system for contact thermal exchange between a pad and a patient at an intravascular catheter administration site, comprising:
a pad as recited in any one of claims 1-10; and,
a fluid circulation unit fluidly interconnectable to said inlet port and said outlet port of said pad, and including a fluid reservoir for containing a fluid and a fluid circulation pump, wherein the fluid circulation pump is operable to circulate the fluid through the pad at a negative pressure from the fluid reservoir and back in to the fluid reservoir to provide for thermal exchange with a patient at an intravascular catheter administration site when said pad is positioned adjacent to the intravascular catheter administration site.

32. The system of claim 31, wherein said fluid circulation unit further comprises:
a heat exchanger interconnected to the fluid reservoir for controlling a temperature of the circulated fluid.

33. The system of claim 32, wherein the fluid circulation unit further comprises:
a controller for controlling operation of the heat exchanger to provide for temperature control of the circulated fluid in a predetermined manner.

34. The system of claim 33, further comprising:
a patient temperature sensor for sensing a temperature of a skin region adjacent to an IV catheter administration site and for providing a patient temperature signal indicative thereof, wherein the controller is provided to utilize the patient temperature signal in providing control signals to the heat exchanger.

35. The system of claim 34, further comprising:
a fluid temperature sensor for sensing a temperature of the circulated fluid and for providing a fluid temperature signal indicative thereof, wherein the controller is provided to utilize the fluid temperature signal in providing control signals to the heat exchanger.

36. The system of claim 35, wherein said controller provides said controls signals to the heat exchanger so as to maintain a temperature of said skin region at a temperature within a predetermined temperature range.
